(19) United States
(12) Patent Application Publication
(10) Pub. No.: US 2004/0053847 A1
(43) Pub. Date: Mar. 18, 2004

(54) Diastereomeric peptitdes and pharmaceutical compositions comprising them

(76) Inventors: Yechiel Shai, Yahud (IL); Ziv Oren, Rishon-Le-Zion (IL); Michal Shahar, Holon (IL); Nurit Eyal, Ness Ziona (IL)

Correspondence Address: BROWDY AND NEIMARK, P.L.L.C., 624 NINTH STREET, NW, SUITE 300, WASHINGTON, DC, 20001-5303, US

(21) Appl. No.: 10/416,789

(22) PCT Filed: Nov. 7, 2001
(86) PCT No.: PCT/IL01/01036

(30) Foreign Application Priority Data
Nov. 16, 2000 (IL) 139720

(51) Int. Cl. A61K 38/10; C07K 7/08; C07K 7/64

(52) U.S. Cl. 514/13; 530/326

(57) ABSTRACT

The invention provides diastereomeric peptides with a net positive charge greater than +1, and cyclic analogues thereof, having at least 15 amino acid residues, wherein said residues are selected from: (i) leucine and lysine;, (ii) leucine, lysine and arginine; (iii) glycine, serine, spargine, threonine, or glutamine optionally present at the N-terminus; (iv) and/or lysine, arginine, glycine, or serine optionally present at the C-terminus. The diastereomeric peptides are suitable for treatment of cancer and for treatment, particularly topical treatment, of infections caused by pathogenic organisms such as bacteria and fungi. In addition, they may be used for controlling, mycoplasma infection in cell cultures and for food preservation as food supplements and as alternative to antibiotics for animal nutrition.

### FIELD OF THE INVENTION

The present invention relates to diastereomeric peptides and to pharmaceutical compositions comprising them.

### BACKGROUND OF THE INVENTION

Antimicrobial resistance to conventional antibiotics has become a major problem worldwide because of their extensive use. In fact, strains of bacteria already exist that are resistant to all available antibiotics.

The development of new families of antibiotics that can overcome the resistance problem has become a very important task. One such major family consists of the antimicrobial peptides. Antimicrobial peptides are natural antibiotics that constitute a major part of the innate immunity of a wide range of organisms. During the past two decades, numerous studies have demonstrated the essential role of antimicrobial peptides as the first line of defense against invading pathogens and their uncontrolled proliferation.

An important property of most antimicrobial peptides is their ability to selectively kill bacteria but not normal eukaryotic cells. Many studies indicated that a specific secondary structure is required and that the common feature found in most of them are an amphipathic character and a net positive charge. Most of the studies were carried out with linear amphipathic α-helical cytolytic peptides (≦40 amino acids) from a host-defense system (Boman, 1995). These polypeptides vary considerably in chain length, hydrophobicity, and overall distribution of charges, but share a common structure upon association with lipid bilayers, namely, an amphipathic α-helical structure. This group includes: (i) lytic peptides that are toxic only to microorganisms, e.g., cecropins isolated from the cecropia moth and magainins and dermaseptins isolated from the skin of frogs; (ii) lytic peptides that lyse mammalian cells, such as δ-hemolysin isolated from S. aureus; and (iii) non-cell-selective lytic peptides such as the bee venom melittin and the neurotoxin pardaxin, that can lyse microorganisms and mammalian cells.

Both pardaxin and melittin are endowed with potent antibacterial activity on Gram-negative and Gram-positive bacteria; however, they also exhibit high hemolytic activity towards human erythrocytes. Recently, we have incorporated several D-amino acids into the α-helical cytolytic peptides pardaxin (Shai and Oren, 1996; International PCT Publication No. WO 97/31019) and melittin (Oren and Shai, 1997; WO 97/31019). Although the resulting diastereomeric analogues of pardaxin and melittin had reduced cytotoxic effects on mammalian cells, they retained high antibacterial activity, thus providing a basis for designing novel peptide antibiotics composed of D- and L-amino acids that are selective to microorganisms.

International PCT Publication WO 98/37090 of the same applicants describes peptides comprising both L- and D-amino acid residues with a net positive charge greater than +1, having a sequence of amino acid residues such that a corresponding amino acid sequence comprising only L-amino acid residues is not found in nature. These non-natural synthetic peptides may be composed of varying ratios of at least one hydrophobic amino acid such as, for example, leucine, isoleucine, glycine, alanine, valine, phenylalanine, proline, tyrosine and tryptophan, and at least one positively charged amino acid such as, for example, lysine, arginine and histidine, in which sequence at least one of the amino acid residues is a D-amino acid. Several diastereomers comprising from 6 to 30 amino acid residues are disclosed in WO 98/37090, but the biological activity was tested only for some 6-mer, 8-mer and 12-mer peptides. Some short-model peptides (12-amino acid long) composed of only leucine and lysine at varying ratios, in which one-third of the sequence consisted of D-amino acids, were further investigated and some of them were found to have reduced hemolytic activity (Hong, Oren & Shai, 1999; Oren, Hong & Shai, 1997).

### SUMMARY OF THE INVENTION

It is an object of the present invention to provide non-natural synthetic diastereomeric peptides that are suitable for topical application against several pathogens.

It is another object of the present invention to provide non-natural synthetic diastereomeric peptides that are suitable for treatment of cancer.

It has now been found that certain diastereomeric peptides not disclosed in any of the references cited above, and in particular not disclosed in the above-mentioned WO 98/37090, have an enhanced activity for topical application and for the treatment of cancer in comparison to the closest diastereomer disclosed in said WO 98/37090.

The present invention thus relates to a diastereomeric peptide having at least 15 amino acid residues selected from leucine and lysine or leucine, lysine and arginine, and optionally at the N-terminus an additional amino acid selected from glycine, serine, aspargine, threonine, and glutamine and/or at the C-terminus an amino acid selected from lysine, arginine, glycine, and serine, with a net positive charge greater than +1, and cyclic analogues thereof.

In one embodiment, the diastereomeric peptide of the invention is composed solely of leucine and lysine residues. Examples of such peptides are the 15-mer peptides herein designated Peptides 2, 3, 4, 7 and 10, of the sequences:

| | |
|---|---|
| 2 | Lys-Leu-Leu-Leu-Lys-Leu-Leu-Lys-Lys-Leu-Leu-Lys-Leu-Leu-Lys-NH₂ |
| 3 | Leu-Lys-Leu-Leu-Lys-Lys-Leu-Leu-Lys-Lys-Leu-Leu-Lys-Leu-Leu-NH₂ |
| 4 | Leu-Lys-Leu-Leu-Lys-Lys-Leu-Leu-Lys-Lys-Leu-Leu-Lys-Leu-Leu-NH₂ |
| 7 | Lys-Leu-Leu-Lys-Leu-Leu-Lys-Lys-Leu-LeuLys-Leu-Leu-Leu-Lys-NH₂ |
| 10 | Lys-Leu-Leu-Leu-Lys-Leu-Leu-Lys-Lys-Leu-Leu-Lys-Leu-Leu-Lys-NH₂ |

In another embodiment, the diastereomeric peptide of the invention is composed of leucine, lysine and arginine residues. Examples of such peptides are the 15-mer peptides herein designated Peptides 6 and 9, of the sequences:

| | |
|---|---|
| 6 | Lys-Leu-Leu-Leu-Arg-Leu-Leu-Lys-Lys-Leu-Leu-Arg-Leu-Leu-Lys-NH₂ |
| 9 | Lys-Leu-Leu-Arg-Leu-Leu-Lys-Lys-Leu-Leu-Arg-Leu-Leu-Leu-Lys-NH₂ |

In a further embodiment, the invention relates to such diastereomeric peptides having at the N-terminus an additional amino acid selected from glycine, serine, aspargine, threonine, and glutamine. In one particular embodiment this additional amino acid is glycine residue, and examples thereof are the 16-mer peptides of the sequences:

| | |
|---|---|
| 5 | Gly-Leu-Leu-Leu-Arg-Leu-Leu-Lys-Lys-Leu-Leu-Arg-Leu-Leu-Lys-Lys-NH₂ |
| 8 | Gly-Leu-Leu-Lys-Leu-Leu-Lys-Lys-Leu-Leu-Lys-Leu-Leu-Leu-Lys-Lys-NH₂ |

In still another embodiment, the invention relates to such diastereomeric peptides having at the C-terminus an amino acid selected from lysine, arginine, glycine, and serine.

In still a further embodiment, the invention relates to cyclic analogues of the diastereomeric peptide. Examples of such cyclic peptides include the peptides of the sequences:

```
  | |
Cys-Lys-Leu-Leu-Leu-Lys-Leu-Leu-Lys-Lys-Leu-Leu-Lys-Leu-Leu-Lys-Cys
  | |
Cys-Leu-Lys-Leu-Leu-Lys Lys-Leu-Leu-Lys-Lys-Leu-Leu-Lys-Leu-Leu-Cys
  | |
Cys-Lys-Leu-Leu-Leu-Arg-Leu-Leu-Lys-Lys-Leu-Leu-Arg-Leu-Leu-Lys-Cys
```

In another aspect, the present invention provides a pharmaceutical composition comprising a diastereomeric peptide of the invention and a pharmaceutically acceptable cancer.

In one embodiment, the invention provides pharmaceutical compositions for topical application, for example for the treatment of topical infections caused by pathogenic organisms such as bacterial infections, particularly infections caused by bacteria resistant to antibiotics, and infections caused by pathogenic fungi. Although all peptides of the invention are contemplated for topical use as antifungal and antibacterial, it is shown hereinafter that the peptides herein designated peptides 2-8 are good candidates for these purposes. In addition, the peptides 2 and 6 were shown to be effective against antibiotic-resistant bacteria.

In another embodiment, the present invention provides pharmaceutical compositions comprising a diastereomeric peptide of the invention for the treatment of cancer. It is contemplated that all peptides of the invention are useful for the treatment of malignant tumors as shown herein for peptides 2, 8, 9 and 10. In particular, peptides 9 and 10 were shown in experiments in vitro and in vivo to be effective against breast, colon, lung, melanoma and prostate tumors.

The diastereomeric peptides of the invention are effective against mycoplasma and can further be used to control/eliminate mycoplasma infection in cell cultures in a method comprising treating the cell culture with said diastereomeric peptide.

The invention further relates to the use of a diastereomeric peptide of the invention for the preparation of a pharmaceutical composition for topical application or of a pharmaceutical composition for the treatment of cancer.

The invention still further relates to a method for the treatment of an infection caused by a pathogenic organism that can be treated by topical application which comprises administering topically to an individual in need thereof a diastereomeric peptide of the invention.

In another embodiment, the invention relates to a method for the treatment of a malignant tumor which comprises administering to an individual in need thereof a diastereomeric peptide of the invention.

### BRIEF DESCRIPTION OF THE DRAWINGS

FIG. 1 is a graph showing tumor growth curve in SCID mice containing human breast MCF-7 cells after administration of peptide 9 of the invention (squares), mitomycin (triangles) and PBS (control, circles).

FIG. 2 is a graph showing body weight curve of SCID mice containing human breast MCF-7 cells after administration of peptide 9 of the invention (squares), mitomycin (triangles) and PBS (control, circles).

FIG. 3 is a graph showing lung weight of C-57 black mice infected with lung carcinoma D-122 cells after administration of peptide 9 of the invention or PBS (control).

### DETAILED DESCRIPTION OF THE INVENTION

The diastereomeric peptides of the invention are cytolytic agents of very low toxicity as evaluated herein in animal models. The results of the skin and eye irritation studies revealed low toxicity of the peptides at concentrations considerably higher than those necessary for their antibacterial, anticancer and antifungal activities.

In order to improve the cytolytic activity of the diastereomeric peptide, the effect of several important parameters such as length, amphipathic organization, the location and number of D-amino acids, additional amino acid residues at the N- and C-termini, and polarity of the diastereomeric peptides on their potency, selectivity and spectrum of activity, were examined. For this purpose we synthesized and structurally and functionally characterized a series of linear cytolytic diastereomers. The peptides were then characterized with regard to their biological activity towards pathogenic cells such as bacteria, cancer cells and fungi, on the one hand, and normal mammalian cells such as NIH-3T3 normal fibroblasts cell line, on the other.

The potency and selectivity of the novel diastereomers of the invention is demonstrated herein in the anticancer, antifungal and antimycoplasma assays. The group of 15- and 16-mer diastereomers shown herein was significantly more active than the prior art 12-mer diastereomers (see peptide 1 in Example 1) against malignant cells, pathogenic fungi and mycoplasma. Furthermore, they were active against the malignant cells at concentrations that are 3-15 lower than the concentrations at which they act against NIH-3T3 normal fibroblasts cells.

In one embodiment of the present invention, the new diastereomeric peptides of the invention are particularly useful for topical application. As used herein, the term “topical” means “pertaining to a particular surface area” and the topical agent applied to a certain area of said surface will affect only the area to which it is applied. Thus any and all applications in which the peptides act locally and not through the blood circulation are encompassed by the present invention.

The high potency, the wide spectrum of activity and the low in vivo toxicity of the model diastereomers of the invention pave the way for their use in topical applications against a wide variety of pathogenic organisms in topical infections. Such applications include, but are not limited to, treatment of acne, fungal infections of the scalp, fungal or bacterial infections related to surgical or traumatic wounds, chronic or poorly healing skin lesions (especially in diabetics), vaginal infection (vaginitis), eye and ear infections and burn wounds, infections of mouth and throat, and localized infections such as chronic pulmonary infections in cystic fibrosis, emphysema or asthma that can be treated with aerosol or other formulation for nasal or pulmonary application.

Topical infections are characterized by opportunistic colonization of a wide range of endogenous and exogenous pathogenic cells. In addition, the treatment of severe wounds such as burns or poorly healing wounds e.g. foot ulcers in diabetes mellitus patients, require long term administration of antibiotics and lead to selection of resistant bacteria such as Streptococcus pyogenes or the methicilin-resistant Staphylococcus aureus. These problems could be overcome by the diastereomeric peptides of the invention due to their wide spectrum of activity and their ability to act against non-resistant and resistant bacteria as demonstrated in the case of peptide no. 2 against Staphylococcus aureus and the methicilin-resistant Staphylococcus aureus. The observed resistance of the diastereomers to proteolytic digestion may enable them to reach the digestive system in intact form and to eliminate there bacterial infections such as chronic gastric mucosal infestation by Helicobacter pylori and intestinal bacterial infections.

The activity of the distereomeric peptides against different strains of fungi indicate their potential use for the treatment of nail fungus such as: (i) onychomycoses, the most current nail infection caused mainly by dermiatophytes, in particular by Trichophyton rubrum, and less frequently by Trichophyton mentagrophytes and Epidermophyton floccosum; (ii) infections caused by mold; and (iii) infections caused by yeasts, particularly Candida albicans, as in chronic paronychia and onycholysis, and chronic mucocutaneous candidosis.

In another embodiment of the invention, the new diastereomeric peptides of the invention are useful as anticancer agents. The observed high potency of the diastereomeric peptides against a variety of malignant cells as shown in the examples herein indicate the existence of a common target for their action. This target is most probably the malignant cell membrane, that has been shown to express higher levels of negatively charged phosphatidylserine than normal mammalian cells (Utsugi et al., 1991). Thus, the diastereomeric peptide may also act against topical and skin cancers such as melanoma, against secondary topical tumors in breast, lung, prostate and colon cancer patients, as well as against basal and squamous cell carcinomas.

The diastereomers of the invention may also be used for food preservation, as food supplements, in veterinary compositions as alternative to antibiotics for animal nutrition, as anti-mycoplasma, antibacterial, and antifungal agents for tissue culture media, and as reagents for transformation/transfection of target cells with desired DNA or RNA molecules.

In summary, the simple model diastercomeric peptides of the invention provide an efficient alternative to the complex sequence of hydrophobic and polar amino acids of known native amphipathic α-helical antimicrobial peptides, required to maintain their monomeric form, selectivity and interface location. Furthermore, by modulating the length, amphipathicity, location and number of D-amino acids and the polarity of the hydrophobic and positively charged amino acids, the membrane selectivity and antipathogenic activity of the desired diastereomeric peptides can be determined. Thus, the increased structural and sequence flexibility of the model diastereomeric antimicrobial peptides of the invention provide important advantages for the design of a repertoire of potent antipathogenic diastereomeric polypeptides for the treatment of diseases.

For topical application, the active components can be formulated with a variety of cosmetically and/or pharmaceutically acceptable carriers. The term “pharmaceutically acceptable carrier” refers to a vehicle which delivers the active components to the intended target and which will not cause harm to humans or other recipient organisms. As used herein, “pharmaceutical” will be understood to encompass both human and animal pharmaceuticals. Useful carriers include, for example, water, acetone, ethanol, ethylene glycol, propylene glycol, butane-1,3-diol, isopropyl myristate, isopropyl palmitate, or mineral oil. Methodology and components for formulation of pharmaceutical compositions are well known, and can be found, for example, in Remington's Pharmaceutical Sciences, Eighteenth Edition, A. R. Gennaro, Ed., Mack Publishing Co. Easton Pa., 1990. The carrier may be in any form appropriate to the mode of delivery, for example, solutions, colloidal dispersions, emulsions (oil-in-water or water-in-oil), suspensions, creams, lotions, gels, foams, mousses, sprays and the like.

The formulation, in addition to the carrier and the antimicrobial components, also can comprise other optional materials which may be chosen depending on the carrier and/or the intended use of the formulation. Additional components include, but are not limited to, antioxidants, chelating agents, emulsion stabilizers, e.g. carbomer, preservatives, e.g. methyl paraben, fragrances, humectants, e.g. glycerine, waterproofing agents, e.g. PVP/Eicosene Copolymer, water soluble film-formers, e.g. hydroxypropyl methylcellulose, oil-soluble film formers, cationic or anionic polymers, and the like.

The antimicrobial components are well-suited for combination with other active components intended for topical application.

The invention will now be described with reference to some non-limiting examples.

### EXAMPLES

Experimental Procedures

(i) Materials

4-Methyl benzhydrylamine resin (BHA) and butyloxycarbonyl (Boc) amino acids were purchased from Calbiochem-Novabiochem Co. (La Jolla, Calif., USA). Other reagents used for peptide synthesis included trifluoroacetic acid (TFA, Sigma), N,N-diisopropylethylamine (DIEA, Sigma), dicyclohexylcarbodiimide (DCC, Fluka), 1-hydroxybenzotriazole (1-HOBT, Pierce), and dimethylformamide (DMF, peptide synthesis grade, Biolab, Ill.). All other reagents were of analytical grade. Buffers were prepared in double-distilled water.

(ii) Peptide Synthesis and Purification

Peptides were synthesized by a solid phase method on 4-methyl benzhydrylamine resin (BHA) (0.05 meq) (Merrifield et. al., 1982; Shai et. al., 1990). The resin-bound peptides were cleaved from the resin by hydrogen fluoride (HF) and, after HF evaporation, and washing with dry ether, extracted with 50% acetonitrile/water. HF cleavage of the peptides bound to BHA resin resulted in C-terminus amidated peptides. Each crude peptide contained one major peak, as revealed by RP-HPLC (reverse phase high-performance liquid chromatography), that was 60-80% pure peptide by weight. The synthesized peptides were further purified by RP-HPLC on a C₁₈ reverse phase Bio-Rad semi-preparative column (250×10 mm, 300 nm pore size, 5-μm particle size). The column was eluted in 40 min, using a linear gradient of 25-60% acetonitrile in water, both containing 0.05% TFA (v/v), at a flow rate of 1.8 ml/min. The purified peptides, which were shown to be homogeneous (˜95%) by analytical HPLC, were subjected to amino acid analysis and electrospray mass spectroscopy to confirm their composition and molecular weight.

(iii) Synthesis of the Cyclic Diastereomers.

The cyclic peptides were synthesized by a solid-phase method as described in section (ii) above, without or with cysteine residues at both the N- and C-termini of the peptides. The cyclization without cystein is carried out by protecting the N-terminal, activating the C-terminal, deprotection of the N-terminal and reaction of the C- and N-terminal groups while still bound to the resin. When the peptide contains cystein residues at both the N- and C-termini, after HF cleavage and RP-HPLC purification, the peptides are solubilized at low concentration in PBS (pH 7.3), and cyclization is completed after 12 h. The cyclic peptides are further purified on RP-HPLC and subjected to amino acid analysis to confirm their composition, and SDS-PAGE to confirm their monomeric state.

### Example 1

Synthesis of 15-mer and 16-mer Linear Diastereomeric Peptides and Cyclic Peptides

The following 15-mer and 16-mer C-amidated diastereomeric peptides 2-10 composed of the hydrophobic amino acid Leu and the positively charged amino acid Lys and, optionally, the positively charged amino acid Arg and/or the N-cap amino acid Gly, and containing 4-5 D-amino acid residues, were synthesized as described in Experimental Procedures, sections (ii) and (iii). Peptide 1 is a 12-mer diastereomer described in the above-mentioned WO 98/37090 and herein used for comparison purposes. The peptides will be represented hereinafter by numerals in bold.

The bold and underlined amino acids are D-amino acids.

| | |
|---|---|
| 1. | [D]-L^{3,4,8,10}-K₄L₈ of the sequence: |
| | Lys-Leu-Leu-Leu-Lys-Leu-Leu-Leu-Lys-Leu-Leu-Lys-NH₂ |
| 2. | [D]-L^{3,13},K^{8,9}-K₆L₉ of the sequence: |
| | Lys-Leu-Leu-Leu-Lys-Leu-Leu-Lys-Lys-Leu-Leu-Lys-Leu-Leu-Lys-NH₂ |
| 3. | [D]-L^{3,8},K^{9,13}-K₆L₉ of the sequence: |
| | Leu-Lys-Leu-Leu-Lys Lys-Leu-Leu-Lys-Lys-Leu-Leu-Lys-Leu-Leu-NH₂ |
| 4. | [D]-L^{3,8},K^{6,9,13}-K₆L₉ of the sequence: |
| | Leu-Lys-Leu-Leu-Lys Lys-Leu-Leu-Lys-Lys-Leu-Leu-Lys-Leu-Leu-NH₂ |
| 5. | [D]-L^{3,6,13},K^{8,9}-GK₄R₂L₉ of the sequence: |
| | Gly-Leu-Leu-Leu-Arg-Leu-Leu-Lys-Lys-Leu-Leu-Arg-Leu-Lys-Lys-NH₂ |
| 6. | [D]-L^{3,6,13},K^{8,9}-K₄R₂L₉ of the sequence: |
| | Lys-Leu-Leu-Leu-Arg-Leu-Leu-Lys-Lys-Leu-Leu-Arg-Leu-Leu-Lys-NH₂ |
| 7. | [D]-L^{3,10,13},K^{7,8}-K₆L₉ of the sequence: |
| | Lys-Leu-Leu-Lys-Leu-Leu-Lys-Lys-Leu-Leu-Lys-Leu-Leu-Leu-Lys-NH₂ |
| 8. | [D]-L^{3,10,13},K^{7,8}-GK₆L₉ of the sequence: |
| | Gly-Leu-Leu-Lys-Leu-Leu-Lys-Lys-Leu-Leu-Lys-Leu-Leu-Leu-Lys-Lys-NH₂ |
| 9. | [D]-L^{3,10,13},K^{7,8}-K₄R₂L₉ of the sequence: |
| | Lys-Leu-Leu-Arg-Leu-Leu-Lys-Lys-Leu-Leu-Arg-Leu-Leu-Leu-Lys-NH₂ |
| 10. | [D]-L^{3,6,13},K^{8,9,13}-K₆L₉ of the sequence: |
| | Lys-Leu-Leu-Leu-Lys-Leu-Leu-Lys-Lys-Leu-Leu-Lys-Leu-Leu-Lys-NH₂ |

The following cyclic amidated diastereomeric peptides 11-13 were prepared:

| | |
|---|---|
| 11. | Cyclic-[D]-L^{3,13},K^{8,9}-K₆L₉ |
| | \| \| |
| | Cys-Lys-Leu-Leu-Leu-Lys-Leu-Leu-Lys-Lys-Leu-Leu-Lys-Leu-Leu-Lys-Cys |
| 12. | Cyclic-[D]-L^{3,8},K^{9,13}-K₆L₉ |
| | \| \| |
| | Cys-Leu-Lys-Leu-Leu-Lys Lys-Leu-Leu-Lys-Lys-Leu-Leu-Lys-Leu-Leu-Cys |
| 13. | Cyclic-[D]-L^{3,6,13},K^{8,9}-K₄R₂L₉ |
| | \| \| |
| | Cys-Lys-Leu-Leu-Leu-Arg-Leu-Leu-Lys-Lys-Leu-Leu-Arg-Leu-Leu-Lys-Cys |

### Example 2

Antibacterial Activity of the Diastereomeric Peptides

The antibacterial activity of the peptides was examined in sterile 96-well plates (Nunc F96 microtiter plates) in a final volume of 100 μl as follows: Aliquots (50 μl) of a suspension containing bacteria at concentration of 1×10⁶ Colony-Forming Units (CFU)/ml in culture LB (Lauria broth) medium were added to 50 μl of water containing the peptide in serial 2-fold dilutions in water. Inhibition of growth was determined by measuring the absorbance at 600 nm with a Microplate autoreader E1309 (Bio-tek Instruments), after an incubation time of 18-20 h at 37° C. Antibacterial activities were expressed as the minimal inhibitory concentration (MIC), the concentration at which 100% inhibition of growth was observed after 18-20 h of incubation. The bacteria used were: Escherichia coli ATCC 25922, Acinetobacter baumannii ATCC 19606, Pseudomonas aeruginosa ATCC 27853, Staphylococcus aureus ATCC 6538P, Enterococcus faecalis ATCC 29212, Enterobacter cloacae ATCC 49141. The antibacterial activity of peptides 2 and 6 was also examined against resistant bacteria: methicilin-resistant Staphylococcus aureus (M4RSA) ATCC 700698 and vancomycin-resistant Enterococcus faecium (VRE) ATCC 700221.

The results for peptides 1-8, summarized in Table 1, reveal that peptides 2-8 of the invention are significantly more potent than the known peptide 1 against most bacteria examined. In addition, peptides 2 and 6 are highly active against the above-mentioned resistant bacteria MRSA and VRE, indicating that these bacteria are not resistant to the diastereomeric peptides.

TABLE 1
Minimal Inhibitory Concentration (μg/ml) of diastereoimeric peptides for bacteria growth

| Peptide | | | | | | | S. aureus | E. faecium |
|---|---|---|---|---|---|---|---|---|
| Designation | E. coli | P. aeruginosa | A. baumannii | E. cloacae | E. faecalis | S. aureus | (MRSA) | (VRE) |
| 1 | 22 | 14 | 31 | 16 | 100 | 28 | N.D. | N.D. |
| 2 | 3.37 | 6.75 | 5 | 9 | 12.5 | 12.5 | 12.5 | 3.37 |
| 3 | 1.5 | 5 | 19 | 9 | 4 | 48 | N.D. | N.D. |
| 4 | 2.9 | 5 | 23 | 11 | 18 | 54 | N.D. | N.D. |
| 5 | 1.2 | 5 | 4 | 6 | 43 | 11 | N.D. | N.D. |
| 6 | 6.75 | 6.75 | 15 | 6.5 | 50 | 18 | 50 | 6.75 |
| 7 | 2.5 | 6 | 4 | 10 | 121 | 10 | N.D. | N.D. |
| 8 | 2.1 | 6 | 5 | 8 | 120 | 7 | N.D. | N.D. |

N.D. = Not determined

### Example 3

Antifungal Activity of the Diastereomers

The antifungal activity of the diastereomers was examined in sterile 96-well plates (Nunc F96 microtiter plates) in a final volume of 200 μL as follows: 100 μl of a suspension containing fungi at concentration of 1×10⁴ Colony-Forming Units (CFU)/ml in culture medium (RPMI 1640, 0.165 M MOPS pH 7.0, with L-glutamine, without NaHCO₃) was added to 100 μl of water containing the peptide in serial 2-fold dilutions in water. The fungi were incubated in the presence of the diastereomeric peptides for 24-48 h at 35° C. in a Binder KB115 incubator under agitation. Growth inhibition was determined by measuring the absorbance at 620 nm with a Microplate autoreader E1309 (Bio-tek Instruments). Antifungal activity is expressed as the minimal inhibitory concentration (MIC), the concentration at which 100% inhibition of growth was observed after the incubation time mentioned above. The fungi used were: Aspergillus niger ATCC 9642, Candida albicans ATCC 10231 and Cryptococcus neoformans ATCC 66031.

The results for peptides 1-8, summarized in Table 2, show that while the known 12-mer peptide 1 was inactive against C. albicans and A. niger, peptides of the invention 2-8 were active against all the fungi examined.

TABLE 2
MIC (μg/ml) of the diastereomeric peptide for fungi growth

| Peptide | C. albicans | C. neoformans | A. niger |
|---|---|---|---|
| Designation | ATCC 10231 | ATCC 66031 | ATCC 9642 |
| 1 | >200 | 4.7 | >200 |
| 2 | 100 | 1.4 | 50 |
| 3 | 40 | 2.8 | 90 |
| 4 | 50 | 5.6 | 180 |
| 5 | 50 | 3 | 50 |
| 6 | 150 | 1.5 | 200 |
| 7 | 50 | 2.9 | 50 |
| 8 | 30 | 1.5 | 30 |

### Example 4

Anticancer Activity of the Diastereomeric Peptides

4a. In vitro Activity Against Mouse Melanoma Cell Line

The anticancer activity of the diastereomers 1, 2, 8, 9 and 10 was examined against B16 F10 mouse melanoma cell line. Melanoma cells were grown in RPMI-1640 medium supplemented with 10% fetal calf serum and antibiotics, at 37° C., in humidified atmosphere at 5% CO₂ and 95% air. In addition, the cell-selectivity of the diastereomeric peptides was studied by examining their effect on NIH-3T3 normal fibroblasts cell line. NIH-3T3 mouse fibroblasts cells were grown in DMEM medium supplemented with 10% bovine calf serum and antibiotics under the same conditions as described above for B16 F10 mouse melanoma cells. Melanoma cells (2×10⁵ cells ml⁻¹) or NIH-3T3 cells (2.5×10⁵ cells ml⁻¹) were seeded in tissue culture plates (3.5 cm). After 48 h, cells were washed thrice with the corresponding medium. Then the cells were incubated at 37° C. in medium (without serum and antibiotics) in the presence of the tested diastereomeric peptides dissolved in PBS, at different concentrations. In control experiments, PBS alone or the chemotherapeutic agent mitomycin C were added to the cells. Following 24 h of incubation, the cells were harvested with trypsin (0.25%)-EDTA (0.05%) and centrifuiged for 10 min at 270×g. The precipitated cells were suspended in the corresponding medium and counted in a hemocytometer (Neubauer). Cell viability was determined by Trypan blue dye exclusion. Anticancer activities were expressed as LC50, the concentration at which 50% cell mortality was observed after 24 h incubation.

The results for peptides 2, 8, 9, and 10, summarized in Table 3, show that the diastereomers 2, 8, 9, and 10 of the invention are significantly more potent than the known 12-mer diastereomer 1 against the melanoma cell line. Furthermore, peptides 2, 8, 9, and 10 were active against melanoma cell line at concentrations which are 3-15 fold lower than the concentration at which they act against NIH-3T3 normal fibroblast cell line. In contrast, both mitomycin C and peptide 1 were active against both cell lines at similar concentration. These results clearly reveal that the new diastereomeric peptides of the invention are more selective than the clinically used chemotherapeutic agent mitomycin C.

TABLE 3
Lethal Concentration (LC50) (μg/ml) of
peptides 1, 2, 8, 9 and 10 against
melanoma and normal fibroblast cell lines

Cell Lines

| Peptide designation | B16-F10 melanoma | NIH-3T3 fibroblasts |
|---|---|---|
| 1 | 90 | 100 |
| 2 | 9 | 30 |
| 8 | 9 | 30 |
| 9 | 2 | 30 |
| 10 | 9 | 140 |
| Mitomycin C | 3 | 3 |

4b. In vitro Activity Against Human Tumor Cell Lines

The anticancer activity of diastereomers 9 and 10 of the invention was also examined against 5 different human tumor cell lines as shown in Table 4 below:

TABLE 4
Human tumor cell lines

| Cell | Source | Type of Cell Line |
|---|---|---|
| MCF-7 | ATCC HTB-22 | Breast adenocarcinoma, pleural effusion, |
| | | human |
| HT-29 | ATCC HTB-38 | Adenocarcinoma, colon, moderately well- |
| | | differentiated grade II, human |
| A 549 | ATCC CCL-185 | Lung carcinoma, human |
| SK-MEL- | ATCC HTB-70 | Malignant melanoma, human |
| PC-3 | ATCC CRL- | Prostate, adenocarcinoma, human |

Aliquots of 100 μl of tumor cell suspension (about 2.5-5×10³/well) were placed in 96-well microtiter plates in an atmosphere of 5% CO₂ at 37° C. After 24 hours, 100 μl of growth medium, 2 μl of test solution, mitomycin or vehicle (PBS, pH=7.4) were added per well in duplicate for an additional 72-hour incubation. The tested diastereomeric peptide was evaluated at concentrations of 100, 10, 1, 0.1 and 0.01 μM. At the end of incubation, 20 μl of AlamarBlue 75% reagent was added to each well for another 6-hour incubation before detection of cell viability by fluorescence intensity. Fluorescence intensity was measured using a Spectraflour Plus plate reader with excitation at 530 nm and emission at 590 nm.

The assays were used to detect changes in cell proliferation based on the ability of viable cells to cause AlamarBlue to change from its oxidized (non-fluorescent, blue) to a reduced (fluorescent, red) form. With the results obtained from the AlamarBlue reaction, cell proliferation can be quantified and metabolic activity of viable cells can be examined.

As can be seen in Table 5, the diastereomers 9 and 10 exhibit significant growth inhibition (>50%) relative to the respective vehicle-treated control group at assay concentrations between about 1 and 20 μM in all the 5 different human tumor cell lines. This shows that the diastereomeric peptides of the invention have a wide spectrum of activity against cancer. The potent activity of the peptides against different types of tumor cell lines and their cell selectivity make them good candidates to be used as anticancer drugs.

TABLE 5
IC50 (μg/ml) of peptides 9 and 10 against human tumor cell lines

| Cell line | Test | *IC₅₀ |
|---|---|---|
| Tumor, Breast, MCF-7 | 10 | 3.6 μM |
| | 9 | 3.3 μM |
| Tumor, Colon, HT-29 | 10 | 15 μM |
| | 9 | 2.2 μM |
| Tumor, Lung, A549 | 10 | 2.4 μM |
| | 9 | 1.4 μM |
| Tumor, Melanoma, SK- | 10 | 2.6 μM |
| MFI 5 | 9 | 1.2 μM |
| Tumor, Prostate, PC-3 | 10 | 8.9 μM |
| | 9 | 1.6 μM |

*IC₅₀ (50% Inhibition Concentration) is the test compound concentration in which the increase from time₀ of the number of treated cells was only 50% as compared to the corresponding increase in the vehicle-(control) treated cells at the end of the experiment.

4c. In vitro Activity Against Breast Cancer

The in-vivo anticancer assay was conducted on SCID (Severe Combined Immune Deficiency) mice using the xenograft model of human breast MCF-7 cells. Groups of 6 SCID female mice weighing 18-20 g (6-week old), bred in an animal isolator (IVC racks under specific pathogen-free (SPF) conditions at 24±1° C., were used. Viable human breast carcinoma MCF-7 cells (ATCC HTB-22−1×10⁷ cells in 0.2 ml of PBS) were injected subcutaneously (s.c.) into the dorsal side of SCID mice. 50 μg/mouse of estradiol benzoate was injected s.c. weekly as a supplement for 4 weeks. When tumor growth reached ≧5 mm in diameter (about day 12), the animals were randomly assigned into groups of six and administration of vehicle and/or test compounds was started. Peptide 9 at 3 mg/kg or vehicle control (PBS, pH=7.4) in a dosing volume 10 ml/kg was administered intravenously (IV) to animals twice a week for 6 doses. Mitomycin at a dose of 2 mg/kg was administered intraperitoneally (IP) twice a week for 5 doses. The tumor size, body weight and the signs of overt animal toxicity after each treatment were recorded and observed. At the end of the experiment, hematological analysis was carried out.

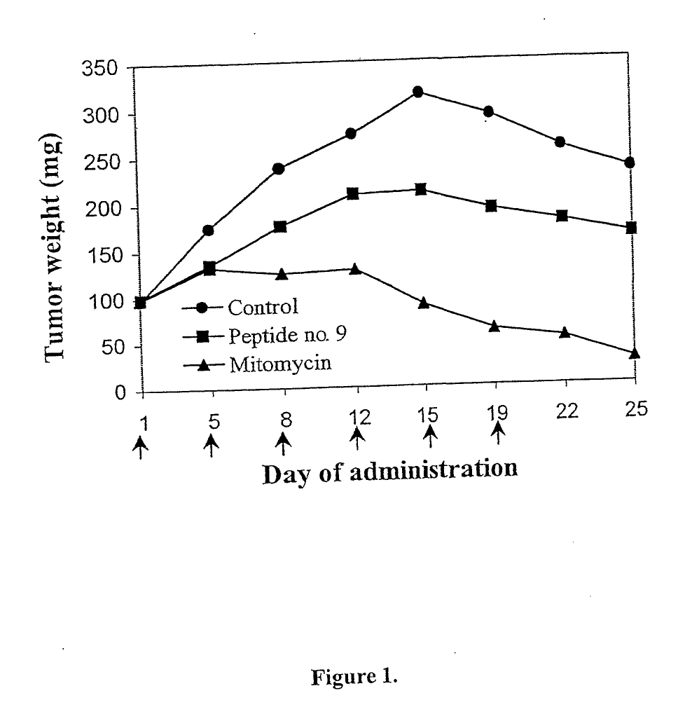

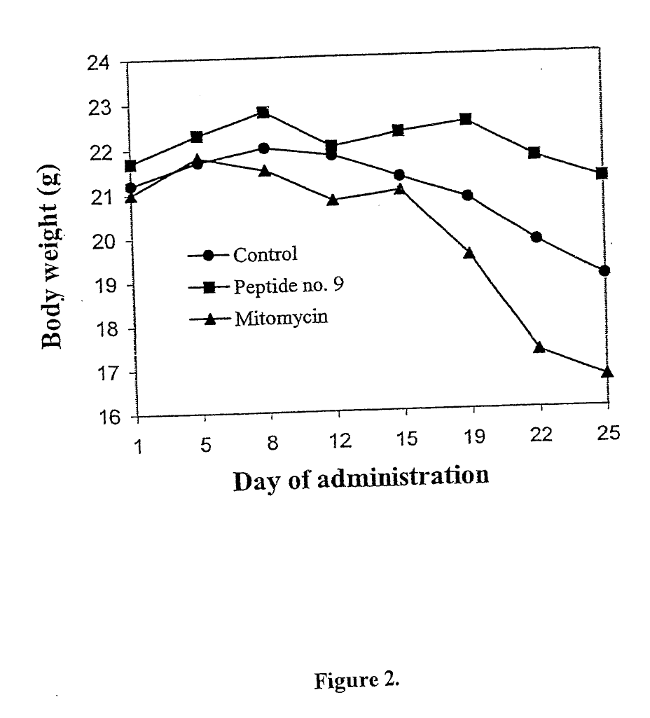

The results summarized in FIG. 1 show that peptide 9 at dosage of 3 mg/kg exhibited inhibitory effect upon the tumor growth over the test period. Although the chemotherapeutic agent mitomycin was more potent than peptide 9 (FIG. 1), the animals treated with mitomycin exhibited severe side effects throughout the assay period, whereas the animals treated with peptide 9 displayed side effects which appeared only after administration of the peptide and disappeared within 1 hour. FIG. 2 shows that the animals treated with mitomycin and even the control mice showed signs of weakness and loss of weight that aggravated as the experiment proceeded. In contrast, the animals treated with peptide no. 9 were in good condition, they did not express any signs of weakness and they maintained their body weight throughout the study period. In addition, hematological analysis that was conducted at the end of the assay revealed that mitomycin also caused a significant reduction of white blood cells (WBC), red blood cells (RBC) and platelets while peptide 9 did not cause a reduction in WBC and platelets, and only a slight reduction in RBC was noted in animals treated with peptide 9 (Table 6).

The results obtained in this experiment indicate that peptide 9 is a potential candidate for use as anticancer drug, probably at higher concentrations than shown herein. In comparison to mitomycin C, peptide 9 was found to be less toxic and to cause only mild temporary side effects, whereas mitomycin C caused severe side effects that persisted for a long period, even few weeks after its last administration.

TABLE 6
Hematology Analysis

| | | | | Hematology analysis on Day 25, | | |
|---|---|---|---|---|---|---|
| | | | | MEAN ± SEM | | |
| | | | | WBC | | PLT |
| Com- | | | | (× | RBC | (× |
| pound | Route | Dose | N | 10³/μl) | (× 10⁶/μl) | 10³/μl) |
| Control | IV | 10 ml/kg × 6 | 6 | 2.95 ± | 11.92 ± 0.12 | 1302 ± |
| | | | | 0.39 | | 52 |
| Peptide | IV | 3 ml/kg × 6 | 6 | 2.22 ± | 11.18 ± 0.11 | 1310 ± |
| No. 9 | | | | 0.22 | | 55 |
| Mito- | IP | 2 ml/kg × 6 | 6 | 0.67 ± | 10.55 ± 0.37 | 396 ± |
| mycin | | | | 0.16 | | 80 |

N = No. of animals.
WBC = White blood cells;
RBC = Red blood cells;
PLT = Platelets.

4d. In vivo Activity Against Lung Cancer

In order to test the capacity of the diastereomers of the invention to inhibit lung metastasis formation, the murine malignant 3LL D-122 Lewis lung carcinoma model (D122 clone of the 3LL carcinoma of C57BL/6 origin) was used with C57 black male mice (Porgador et al., 1993). The animals were bred in an animal isolator (IVC racks) under specific pathogen-free (SPF) conditions at 24±1° C. Malignant 3LL D-122 cells (1×10⁶ cells/0.2 ml PBS) were injected intravenously (i.v.) to twenty 8-9 week-old C57 black male mice weighing 18-22 g. After 24 hours, the animals were randomly assigned into two groups and administration of vehicle (control) or of a test compound was started. Peptide 9 at 5 mg/kg or control (PBS, pH=7.4) at 10 ml/kg was administered i.v. every day to the mice for three days in the first week, and then once a week in the next two weeks, for a total of 5 treatments in a period of 21 days. On day 28, mice were sacrificed and their lungs were removed and weighed in order to measure the extent of lung metastasis. The metastatic load is defined as the mean lung weight of mice infected with D-122 cells minus the mean lung weight of 5 normal mice (158±2 mg).

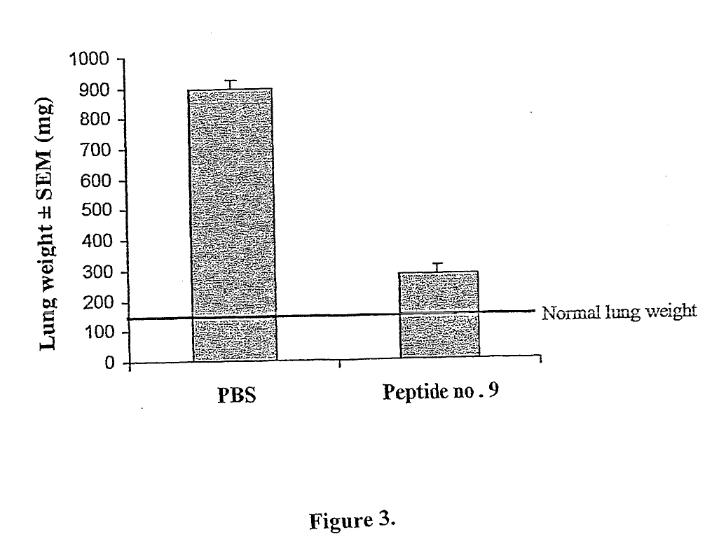

The results are depicted in FIG. 3. As shown, mice infected with D-122 lung carcinoma cells and treated with peptide 9 of the invention exhibited significantly lower lungs weight (282±28 mg) in comparison with D-122 infected and untreated mice (896±39 mg). The metastatic load was decreased by 80% in the group treated with Peptide 9 compared to the untreated group. These findings reveal that peptide 9 is also significantly active against lung tumor metastases and can be used as a broad spectrum anticancer drug.

### Example 5

Resistance of the Diastereomers to Proteolytic Digestion

In order to reach their target, the diastereomers have to withstand proteolytic digestion by proteases which may occur during the time of their administration site till they reach the target site. The susceptibility of the diastereomer 2 of the invention Is to proteolytic digestion by pepsin (from porcine stomach mucosa, Sigma), trypsin (from bovine pancreas, Sigma), and elastase (from human leukocytes, Sigma) was assessed by reverse-phase HPLC. As a control we used the native, all L-amino acid antimicrobial peptide cecropin B. Equal amounts of the peptides were dissolved in PBS (35 mM phosphate buffer/0.15 M NaCl, pH 7.3) at final concentration of 140 μM to which 25 μM of protease were added. The samples were incubated under agitation for 30 min at 37° C. After the addition of the appropriate protease inhibitor to stop the reaction, aliquots were injected to C₁₈ column and the amounts of the intact diastereomers were evaluated using their absorbance at 215 nm. The results summarized in Table 7 show that the diastereomers of the invention are significantly less susceptible to protease digestion as compared to the native antimicrobial peptide cecropin B.

TABLE 7
Proteolytic digestion (%) of the diastereomers to

| Peptide designation | Trypsin | Pepsin | Elastase |
|---|---|---|---|
| 2 | 32 | 10 | 6 |
| Cecropin B | 100 | 100 | 100 |

### Example 6

Activity of the Diastereomers of the Invention Against Mycoplasma

Mycoplasmas are the smallest free-living microorganisms, being about 300 nm in diameter. They are bounded by a triple-layered membrane and, unlike conventional bacteria, do not have a rigid cell wall. Hence, they are not susceptible to penicillins and other antibiotics that act on bacteria. Mycoplasma infection of cell cultures is widespread and has major detrimental effects on cellular physiology and metabolism. Since cell culture is used extensively, both in research and in industrial production processes, it is essential to find a way to eliminate mycoplasma contamination.

In order to examine the activity of the diastereomers of the invention against mycoplasma, 500 μl of Mycoplasma pneumoniae cells were introduced into a cell culture flask containing 50 ml of SP-4 growth media (Jacob et al., 1985). The cells were grown at 37° C. for 3-4 days. Before the assay, the mycoplasma cells were washed 3 times with serum-free medium (without fetal calf serum) and then harvested into 5 ml of the same medium. The cells' concentration was adjusted to approximately 1×10⁶ cells ml⁻¹. Thereafter, 150 μl of either diastereomer 1 or 2 in phosphate-buffered saline (PBS) were added to 150 μl mycoplasma cells. The mixture was incubated for 1 hour at room temperature. As a control, cells were incubated with PBS alone. At the end of the incubation period, 150 μl of the mixture were inoculated into tissue culture flasks (in duplicate) containing 15 ml SP-4 media. After 3-4 days, the mycoplasma viability was determined by counting the number of cells grown in each flask. Anti-mycoplasma activity is expressed as the minimal inhibitory concentration (MIC), the concentration at which 100% inhibition of growth was observed after the incubation time mentioned above.

As shown in Table 8, the 12-mer diastereomer 1 known from the prior art was less active against mycoplasma than the 15-mer peptide 2 of the present invention. Moreover, compared to their activity against normal cell line (Table 3), both peptides were active against NIH-3T3 fibroblasts at concentrations that are 2-fold higher than the concentration at which they act against mycoplasma, indicating that the diastereomeric peptides have a selective activity against mycoplasma.

TABLE 8
MIC (μg/ml) of peptides 1 and 2 against Mycoplasma

| Peptide | |
|---|---|
| designation | Mycoplasma |
| 1 | 50 |
| 2 | 12.5 |

### Example 7

Effect of Diastereomers of the Invention on Primary Skin and Eye Irritation in Rabbits

The purpose of this experiment was to provide information on the irritation likely to arise from a single instillation of peptide 2 into the eye or from a single topical exposure to it. The study was conducted by the service company “PSL” (Product Safety Labs, 725 Cranbury Road, East Brunswick, N.J., USA).

For the eye irritation assay, three healthy rabbits (1 male and 2 females) without pre-existing ocular irritation were selected. Prior to instillation, peptide 2 was dissolved in 0.9% saline to result in a 0.5% concentration, then mixed well using a vortex mixer. One-tenth of a milliliter of the prepared test substance was instilled into the conjunctival sac of the right eye of each rabbit by pulling the lower lid away from the eyeball. The upper and lower lids were then gently held together for about one second before releasing, to minimize loss of the test substance. The left eye remained untreated and served as a control. Ocular irritation was evaluated with the illumination of a white light source at 1, 24, 48 and 72 hours after the instillation according to the “Scale for scoring ocular lesions” used in “PSL”. Fluorescein dye was used at 24 hours to verify the absence of corneal damage.

For the skin irritation assay, three healthy rabbits were selected. On the day before application, the fur on the treatment site of each animal was removed by clipping. Care was taken to avoid abrading the skin. After clipping and prior to application, the animal's skin was checked for any abnormalities according to the “Primary Skin Irritation Scoring System” used in “PSL”. If any site scores were greater than zero, that animal was removed from test and replaced. Prior to the exposure, peptide 2 was dissolved in 0.9% saline to result in a 0.5% concentration, then mixed well using a vortex mixer. Five-tenths of a milliliter of the prepared test substance was applied directly to the skin of the treatment site. The entire trunk of each animal was then wrapped with non-irritating occlusive adhesive tape to avoid dislocation of the patch. Animals were exposed to the test substance for a period of 24 hours. Elizabethan collars were placed on each rabbit for the designated exposure period. Following the exposure period, the patches were removed and the treatment sites were wiped with water or appropriate solvents using clean towels to remove any residual test substance. The treatment sites were examined for signs of erythema and edema within 1 hour and at approximately 24, 48 and 72 hours after patch removal according to the “Primary Skin Irritation Scoring System” developed by Draize (Draize et al., 1944). Individual scores were recorded for each rabbit. A narrative description of pertinent skin observations and any signs of gross toxicity were recorded. In addition, evaluation of the reversibility or irreversibility of the observed effects was noted.

According to the results obtained, no corneal opacity or iritis was noted during the eye irritation study and no signs of erythema and edema were observed during the skin irritation assay. Thus, it can be concluded that peptide 2 does not cause eye or skin irritation at the high concentration tested (0.5%; 5 mg/ml).

### Example 8

Liposome Encapsulation of the Diastereomeric Peptides

Liposomes serve as convenient delivery vehicles for biologically active molecules. Hydrophilic drugs can be encapsulated in the internal aqueous compartment, whereas hydrophobic drugs may bind to or are incorporated in the lipid bilayers. In this experiment, liposomal diastereomeric peptides were prepared in order to lower the peptide toxicity and increase their selectivity.

Liposomes composed of different ratios of phosphatidylcholine (PC)/phosphatidylglycerol (PG) (9:1; 4:1; 1:1 w/w) or phosphatidylethanolamine (PE)/PG (9:1; 4:1; 1:1 w/w) were prepared. In brief, dry lipid mixtures were dissolved in CHCl₃/MeOH (2/1, v/v). The solvents were evaporated under nitrogen stream, and the lipid mixtures at the compositions described above were resuspended in PBS by vortex mixing. The lipid suspension was extruded through 3 different polycarbonate filters (1 μm, 0.2 μm and 0.1μμ pore size filters, 15 times each). Finally, the resulting suspensions of large unilamellar vesicles (LUV) were added to different concentrations of peptide 9 to give lipid/peptide ratios of 50:1; 30:1; 10:1 w/w, respectively. The mixtures were sonicated for 2 minutes and the liposomes were stored at 4° C. until used.

The antibacterial activity of the resulting liposomal diastereomeric peptide preparations were examined as described in Example 2 above. The MICs of liposomal peptide 9 in various lipid compositions and peptide/lipid ratios (as described above), or of liposomes at lipid compositions equivalent to the loaded_liposomes (not shown) or peptide 9 alone, were determined using the following bacteria: Acinetobacter baumannii ATCC 19606, and Staphylococcus aureus ATCC 6538P.

The results summarized in Table 9 show that liposomal peptide 9 composed of the lipids PC/PG (9:1 w/w) at a 10:1 w/w lipid/peptide ratio, exhibits MIC results similar to those of peptide 9 alone. Hence, peptide 9 entrapped within liposomes can maintain its antibacterial activity, however this activity is contingent on the liposomes' lipid composition and on the lipid/peptide ratio.

TABLE 9
MIC (μg/ml) of liposomal peptide 9 for bacteria

| | lipid/peptide | | |
|---|---|---|---|
| Liposome | ratio | | |
| composition (W/W) | (W/W) | A. baumannii | S. aureus |
| PC/PG 4:1 | 30:1 | 6.25 | 50 |
| PC/PG 9:1 | 50:1 | 3.1 | 50 |
| PC/PG 4:1 | 50:1 | 12.5 | 50 |
| PC/PG 9:1 | 10:1 | 3.1 | 12.5 |
| | Peptide 9 alone | 3.1 | 12.5 |

The in vivo toxicity of the liposomal peptide 9 preparation composed of the lipids PC/PG (9:1 w/w) at a 10:1 w/w lipid/peptide ratio was examined. Groups of 5 CD1 male mice weighing 24-27 g (5-week old), bred in an animal isolator (IVC racks) under specific pathogen-free (SPF) conditions at 24±1° C., were used. Twelve mg/kg liposomal peptide 9 or peptide 9 alone dissolved in PBS in a dosing volume of 10 ml/kg, were administered by single i.v. bolus injection via the mice tail vein. In parallel, control groups received i.v. injections of equivalent liposomes alone or PBS in a dosing volume of 10 ml/kg.

The results in Table 10 show that 12 mg/kg of peptide 9 injected i.v. caused 80% mortality, while liposomal peptide 9 in the same concentration produced only 20% mortality. No incidence of mortality occurred following the i.v. injection of PBS or the liposomes alone. These results show that entrapment of diastereomeric peptides of the invention in liposomes can reduce their toxicity while maintaining their activity.

TABLE 10
Incidence of mortality in mice following single i.v. injection of
liposomal peptide 9, peptide 9, liposomes, or PBS.

| | Incidence of |
|---|---|
| Treatment | mortality* |
| Liposomal peptide 9 | 1/5 |
| (12 mg/kg) | |
| Peptide 9 (12 mg/kg) | 4/5 |
| Liposomes | 0/5 |
| PBS | 0/5 |

*No. animals affected/No. animals per group

References

Draize J. H., Woodward, G. & Calvery, H. O. (1944). J Pharmacol Exp Ther 82, 377-390.

Hong, J., Oren, Z. & Shai, Y. (1999). Biochemistry.

Jacob, E., Schopperele, K. & Bredt, W. (1985). Eur. J. Clin. Microbiol. 4, 113-118.

Oren, Z. & Shai, Y. (1996). Biochemistry 36, 1826-1835.

Oren, Z. & Shai, Y. (2000). Biochemistry 39, 6103-6114.

Oren, Z., Hong, J. & Shai, Y. (1997). J. Biol. Chem. 272, 14643-14649.

Porgador, A., Bannerj, R., Watanabe, Y., Feldman, M., Gilboa, E. & Eisenbach, L (1993). J. Immunol. 150, 1458-1470.

Raff, M. C. (1971). Transpl. Rev. 6, 52-80.

Shai, Y. & Oren, Z. (1996). J. Biol. Chem. 271, 7305-7308.

Sharon, M., Oren, Z., Shai, Y. & Anglister, J. (1999). Biochemistry 38, 15303-15316.

Utsugi, T., Schroit, A. J., Connor, J., Bucana, C. D. & Fidler, I. J. (1991). Cancer Res. 51, 3062-3066.

1. A diastereomeric peptide with a net positive charge greater than +1, and cyclic s analogues thereof, having at least 15 amino acid residues, wherein said residues are selected from: (i) leucine and lysine; (ii) leucine, lysine and arginine; (iii) glycine, serine, aspargine, threonine, or glutamine optionally present at the N-terminus; (iv) and/or lysine, arginine, glycine, or serine optionally present at the C-terminus.

2. A diastereomeric peptide according to claim 1 composed of leucine and lysine residues.

3. A diastereomeric peptide according to claim 2, selected from the peptides of the sequences:

| | |
|---|---|
| Lys-Leu-Leu-Leu-Lys-Leu-Leu-Lys-Lys-Leu-Leu-Lys-Leu-Leu-Lys-NH₂ | 7 |
| Leu-Lys-Leu-Leu-Lys-Lys-Leu-Leu-Lys-Lys-Leu-Leu-Lys-Leu-Leu-NH₂ | 3 |
| Leu-Lys-Leu-Leu-Lys-Lys-Leu-Leu-Lys-Lys-Leu-Leu-Lys-Leu-Leu-NH₂ | 4 |
| Lys-Leu-Leu-Lys-Leu-Leu-Lys-Lys-Leu-Leu-Lys-Leu-Leu-Leu-Lys-NH₂ | 7 |
| Lys-Leu-Leu-Leu-Lys-Leu-Leu-Lys-Lys-Leu-Leu-Lys-Leu-Leu-Lys-NH₂ | 10 |

4. A diastereomeric peptide according to claim 1 composed of leucine, lysine and arginine residues.

5. A diastereomeric peptide according to claim 4, selected from the peptides of the sequences:

| | |
|---|---|
| Lys-Leu-Leu-Leu-Arg-Leu-Leu-Lys-Lys-Leu-Leu-Arg-Leu-Leu-Lys-NH₂ | 6 |
| Lys-Leu-Leu-Arg-Leu-Leu-Lys-Lys-Leu-Leu-Arg-Leu-Leu-Leu-Lys-NH₂ | 9 |

6. A diastereomeric peptide according to claim 1 having at the N-terminus of the peptide an amino acid selected from glycine, serine, aspargine, threonine, and glutamine.

7. A diastereomeric peptide according to claim 6 wherein said amino acid is glycine.

8. A diastereomeric peptide according to claim 7, selected from the peptides of the sequences:

| | |
|---|---|
| Gly-Leu-Leu-Leu-Arg-Leu-Leu-Lys-Lys-Leu-Leu-Arg-Leu-Leu-Lys-Lys-NH₂ | 5 |
| Gly-Leu-Leu-Lys-Leu-Leu-Lys-Lys-Leu-Leu-Lys-Leu-Leu-Leu-Lys-Lys-NH₂ | 8 |

9. A diastereomeric peptide according to claim 1 having at the C-terminus an amino acid selected from lysine, arginine, glycine, and serine.

10. A diastereomeric peptide according to claim 1 wherein the peptide is a cyclic analogue of said diastereomeric peptide.

11. A diastereomeric peptide according to claim 10, selected from the peptides of the sequences:
```
  | |
Cys-Lys-Leu-Leu-Leu-Lys-Leu-Leu-Lys-Lys-Leu-Leu-Lys-Leu-Leu-Lys-Cys
  | |
Cys-Leu-Lys-Leu-Leu-Lys Lys-Leu-Leu--Lys-Lys-Leu-Leu-Lys-Leu-Leu-Cys
  | |
Cys-Lys-Leu-Leu-Leu-Arg-Leu-Leu-Lys-Lys-Leu-Leu-Arg-Leu-Leu-Lys-Cys
```

12. A pharmaceutical composition comprising a diastereomeric peptide according to claim 1 and a pharmaceutically acceptable carrier.

13. A pharmaceutical composition according to claim 12 for topical application.

14. A pharmaceutical composition according to claim 12 or 13, for topical treatment of acne, fungal infections of the scalp, fungal or bacterial infections related to surgical or traumatic wounds, chronic or poorly healing skin lesions such as foot ulcer in diabetes mellitus patients, vaginal infection (vaginitis), eye and ear infections and burn wounds, infections of mouth and throat, and localized infections such as chronic pulmonary infections in cystic fibrosis, emphysema and asthma.

15. A pharmaceutical composition according to claim 12 or 13 for the treatment of topical infections caused by pathogenic organisms.

16. A pharmaceutical composition according to claim 15 for the treatment of bacterial infections.

17. An antibacterial pharmaceutical composition according to claim 16 comprising a diastereomeric peptide selected from the peptides of the sequences:

| | |
|---|---|
| Lys-Leu-Leu-Leu-Lys-Leu-Leu-Lys-Lys-Leu-Leu-Lys-Leu-Leu-Lys-NH₂ | 2 |
| Leu-Lys-Leu-Leu-Lys-Lys-Leu-Leu-Lys-Lys-Leu-Leu-Lys-Leu-Leu-NH₂ | 3 |
| Leu-Lys-Leu-Leu-Lys-Lys-Leu-Leu-Lys-Lys-Leu-Leu-Lys-Leu-Leu-NH₂ | 4 |
| Lys-Leu-Leu-Leu-Arg-Leu-Leu-Lys-Lys-Leu-Leu-Arg-Leu-Leu-Lys-NH₂ | 6 |
| Lys-Leu-Leu-Lys-Leu-Leu-Lys-Lys-Leu-Leu-Lys-Leu-Leu-Leu-Lys-NH₂ | 7 |
| Gly-Leu-Leu-Leu-Arg-Leu-Leu-Lys-Lys-Leu-Leu-Arg-Leu-Leu-Lys-Lys-NH₂ | 5 |
| Gly-Leu-Leu-Lys-Leu-Leu-Lys-Lys-Leu-Leu-Lys-Leu-Leu-Leu-Lys-Lys-NH₂ | 8 |

18. A pharmaceutical composition according to any one of claims 12 to 17 or the treatment of chronic gastric mucosal infestation by Helicobacter pylori and intestinal bacterial infections.

19. A pharmaceutical composition according to claim 16 for the treatment of infections caused by antibiotic-resistant bacteria.

20. A pharmaceutical composition according to claim 19 comprising a diastereomeric peptide selected from the peptides of the sequences:

| | |
|---|---|
| Lys-Leu-Leu-Leu-Lys-Leu-Leu-Lys-Lys-Leu-Leu-Lys-Leu-Leu-Lys-NH₂ | 2 |
| Lys-Leu-Leu-Leu-Arg-Leu-Leu-Lys-Lys-Leu-Leu-Arg-Leu-Leu-Lys-NH₂ | 6 |

21. A pharmaceutical composition according to claim 19 or 20 wherein said resistant bacteria are selected from Streptococcus pyogenes and the methicilin-resistant Staphylococcus aureus.

22. A pharmaceutical composition according to claim 15 for the treatment of fungal infections.

23. An antifungal pharmaceutical composition according to claim 22 comprising a diastereomeric peptide selected from the peptides of the sequences:

| | |
|---|---|
| Lys-Leu-Leu-Leu-Lys-Leu-Leu-Lys-Lys-Leu-Leu-Lys-Leu-Leu-Lys-NH₂ | 2 |
| Leu-Lys-Leu-Leu-Lys-Lys-Leu-Leu-Lys-Lys-Leu-Leu-Lys-Leu-Leu-NH₂ | 3 |
| Leu-Lys-Leu-Leu-Lys-Lys-Leu-Leu-Lys-Lys-Leu-Leu-Lys-Leu-Leu-NH₂ | 4 |
| Lys-Leu-Leu-Leu-Arg-Leu-Leu-Lys-Lys-Leu-Leu-Arg-Leu-Leu-Lys-NH₂ | 6 |
| Lys-Leu-Leu-Lys-Leu-Leu-Lys-Lys-Leu-Leu-Lys-Leu-Leu-Leu-Lys-NH₂ | 7 |
| Gly-Leu-Leu-Leu-Arg-Leu-Leu-Lys-Lys-Leu-Leu-Arg-Leu-Leu-Lys-Lys-NH₂ | 5 |
| Gly-Leu-Leu-Lys-Leu-Leu-Lys-Lys-Leu-Leu-Lys-Leu-Leu-Leu-Lys-Lys-NH₂ | 8 |

24. A pharmaceutical composition according to claim 22 or 23, for topical treatment of nail fungi, infections caused by yeasts such as Candida albicans, fungal infections of the scalp, and fungal infections related to surgical or traumatic wounds.

25. A pharmaceutical composition according to any one of claims 12 to 24 in the form of solution, colloidal dispersion, cream, lotion, gel, foam, mousse, spray, aerosol or other formulation for nasal or pulmonary application.

26. A pharmaceutical composition according to claim 12 for treatment of cancer.

27. An anticancer pharmaceutical composition according to claim 26 comprising a diastereomeric peptide selected from the peptides of the sequences:

| | |
|---|---|
| Lys-Leu-Leu-Leu-Lys-Leu-Leu-Lys-Lys-Leu-Leu-Lys-Leu-Leu-Lys-NH₂ | 2 |
| Gly-Leu-Leu-Lys-Leu-Leu-Lys-Lys-Leu-Leu-Lys-Leu-Leu-Leu-Lys-Lys-NH₂ | 8 |
| Lys-Leu-Leu-Arg-Leu-Leu-Lys-Lys-Leu-Leu-Arg-Leu-Leu-Leu-Lys-NH₂ | 9 |
| Lys-Leu-Leu-Leu-Lys-Leu-Leu-Lys-Lys-Leu-Leu-Lys-Leu-Leu-Lys-NH₂ | 10 |

28. A pharmaceutical composition according to claim 26 or 27 for treatment of breast, colon, lung, melanoma or prostate tumors.

29. A pharmaceutical composition according to claim 28 comprising a diastereomeric peptide selected from the peptides of the sequences:

| | |
|---|---|
| Lys-Leu-Leu-Arg-Leu-Leu-Lys-Lys-Leu-Leu-Arg-Leu-Leu-Leu-Lys-NH₂ | 9 |
| Lys-Leu-Leu-Leu-Lys-Leu-Leu-Lys-Lys-Leu-Leu-Lys-Leu-Leu-Lys-NH₂ | 10 |

30. A pharmaceutical composition according to claim 26 for treatment of melanoma, and basal and squamous cell carcinomas.

31. A pharmaceutical composition according to any one of claims 12 to 30, in the form of a liposomal preparation.

32. A composition comprising a diastereomeric peptide according to claim 1 for control of mycoplasma infection in cell cultures.

33. A composition comprising a diastereomeric peptide according to claim 1 for food preservation.

34. A composition comprising a diastereomeric peptide according to claim 1 for use as food supplement.

35. A veterinary composition comprising a diastereomeric peptide according to claim 1.
